(19) United States
(12) Patent Application Publication
(10) Pub. No.: US 2007/0250994 A1
(43) Pub. Date: Nov. 1, 2007

(54) Goggle having flexible headband attachment arms fixedly attached to a goggle frame

(76) Inventors: Joseph R. McNeal, Hailey, ID (US); David Henry Currier, Boulder, CO (US)

Correspondence Address: Edward W. Bulchis, Esq.;DORSEY & WHITNEY LLP, Suite 3400, 1420 Fifth Avenue, Seattle, WA, 98101, US

(21) Appl. No.: 11/335,165

(22) Filed: Jan. 18, 2006

(51) Int. Cl. A61F 9/02

(52) U.S. Cl. 2/426

(57) ABSTRACT

A sports goggle having generally U-shaped attachment arms at opposite sides of a goggle frame for securing the goggle frame to an elastic headband. The attachment arms are secured to the goggle frame at locations inward from the sides of the frame. As a result, the attachment arms bend rearwardly so that they are received in respective recesses in the frame when the goggle is worn without a helmet. When the goggle is worn with a helmet, the attachment arms are bent forwardly. However, since the headband extends from the attachment arms with a rearward component, the headband forces the goggle frame against the face of a wearer without creating gaps between the frame and the face of the wearer.

### TECHNICAL FIELD

This invention relates to protective goggles typically used for sporting activities like skiing and snowboarding.

### BACKGROUND OF THE INVENTION

Goggles are widely used in a variety of applications, such as when skiing and snowboarding, working in hazardous conditions or with hazardous substances. Goggles typically include a frame having a somewhat oval configuration with an inner edge adapted to a wearer's face, often through a cushioning foam face gasket. The frame usually has an arcuate shape curving rearwardly at the sides to conform to a wearer's face. A lens is normally mounted in the frame near an outer edge of the frame so that the lens and wearer's face substantially enclose the interior of the frame. The goggle is normally held on the wearer's head by an elastic headband having its ends attached to opposite side edges of the goggle frame. The headband may be attached directly to the goggle frame, or it may be attached through an intervening structure.

In some cases, such as in skiing and snowboarding, goggles are alternately worn with and without a helmet. Most goggles are arranged so that they can be comfortably and effectively worn without a helmet. When a goggle is worn without a helmet, the headband extends from the side edges of the goggle rearwardly along the sides of the wearer's head. In this configuration, the force exerted by the headband pulls the side edges of the frame rearwardly to securely hold the inner edge of the frame against the face of the wearer. However, when some goggles are worn with a helmet, the headband can extend laterally to the forward side edges of the helmet. In this configuration, the force exerted by the headband can pull the sides of the goggle away from the wearer's face, forming gaps between the frame and the wearer's face. These gaps can allow light, air or debris such as snow to enter the inside of the goggle.

A number of techniques have been tried to allow a goggle to be comfortably and effectively worn either with or without a helmet. One approach has been to attach the headband to rigid “outriggers” that project laterally outwardly from the front of the frame. The outriggers relocate the point of attachment between the headband and the frame forwardly and inwardly from the side edges of the frame. By effectively connecting the headband to the front of the frame at locations inwardly from the side edges, the headband is more likely to extend rearwardly when worn with a helmet. As a result, the headband can pull the frame rearwardly to hold the goggle against the face of the wearer. The use of an outrigger does somewhat alleviate the problem of the headband pulling the goggle away from the wearer's face when the goggle is worn with a helmet. However, the outwardly projecting outriggers are considered by some to be unattractive. Furthermore, since the goggle frame should be made from a somewhat soft, resilient material and the outrigger should be made from a somewhat rigid material, two different materials must be used, and it is thus not possible to form the frame and outrigger as an integral unit.

One solution that largely solves the headband angle problem without adversely affecting the appearance of a goggle frame is shown in U.S. Pat. No. 6,732,383 to Cleary et al. In the Cleary et al. goggle, a headband attachment arm is pivotally mounted to each side of the goggle frame at locations spaced inwardly from the edge of the frame. More specifically, pivot pins project inwardly toward each other from opposite ends of each pivot arm, and the ends of the headband are attached to the middle of the arms. The pivot pins are received by respective pivot holes formed on upper and lower surfaces of the frame, respectively. The periphery of the frame is recessed between the pivot holes so that the arms do not project substantially beyond the outer surface of the frame. As a result, the arms and frame together form a smooth contour that provides an attractive appearance and good aerodynamic performance.

In use, the headband attachment arms of the Cleary et al. goggle are recessed within the frame when the goggle is worn without a helmet, and the headband therefore extends rearwardly from the goggle frame. In this configuration, the attachment arms blend in with the goggle frame and are almost invisible. When the goggle is worn with a helmet, the headband extends from the frame more laterally than rearwardly. However, the attachment arms can pivot forwardly to prevent the headband from pulling the frame forwardly away from the face of the wearer.

The Cleary et al. goggle has most of the advantages of the “outrigger” design without some of its disadvantages. However, it is still less than ideal. In particular, the frame must be made relatively thick in the area where the headband attachment arms are pivotally attached to the frame, which can present an unattractive appearance. If, for example, the frame was too thin, the pivot holes would be relatively shallow, thereby potentially allowing the pivot pins to be easily pulled from the holes. Other pivot structures would likewise require the frame to be sufficiently thick to form the pivot structure. Furthermore, the need to form a pivotal connection between the headband attachment arms and frame limits the choices of materials that can be used to form the arms and frame. In particular, the arms must be rigid enough to substantially retain their shape when they are subjected to forces from the headband. Also, in general, the frame must be sufficiently rigid that the pivot structure retains its shape. For example, if the pivot holes were to become elongated or the upper and lower portions of the frame moved toward each other, the pivot pins could easily be pulled from the pivot holes.

Another disadvantage of the Cleary et al. goggle is the added cost of forming attachment arms separately from the frame and possibly using a different material, as well as the assembly cost of attaching the arms to the frame.

There is therefore a need for a goggle that can be worn either with or without a helmet, a goggle that is not pulled away from the face when worn with a helmet, and a goggle that is relatively inexpensive to manufacture and attractive in appearance whether worn with or without a helmet.

### SUMMARY OF THE INVENTION

A goggle that can be worn with or without a helmet includes a frame having an outer periphery surrounding an opening in which a lens is mounted. The frame also includes a top, a bottom and opposite sides. The goggle further includes a pair of generally U-shaped attachment arms extending along respective sides of the frame. Each of the attachment arms has first and second ends that are fixedly attached to the top and bottom of the outer periphery of the frame at attachment locations that are spaced inwardly from the respective side of the frame. Each of the arms has at least one resilient region structured to allow the arm to bend forwardly and rearwardly. A head support such as an elastic headband is attached to the attachment arms to secure the goggle to the head of a wearer. When the goggle is worn without a helmet, the head support extends from the attachment arms substantially rearwardly at a first angle. In this position, the attachment arms are bent rearwardly to a first position. When the goggle is worn with a helmet, the head support extends from the attachment arms at a second angle that is substantially forward of the first angle. In this position, the attachment arms are bent to a second position that is forward of the first position. However, since the head support has a rearward component as it extends from the attachment arms, the goggle frame is held against the face of the wearer without creating any gaps between the frame and the face of the wearer at the sides of the goggle.

### BRIEF DESCRIPTION OF THE DRAWINGS

FIG. 1 is a front elevational view of a goggle according to one example of the invention.

FIG. 2 is top plan view of the goggle of FIG. 1.

FIG. 3 is bottom plan view of the goggle of FIG. 1.

FIG. 4 is side elevational view of the goggle of FIG. 1.

FIG. 5 is a cross-sectional view taken along a horizontal plane extending through the head of a wearer showing the goggle of FIG. 1 worn without a helmet.

FIG. 6 is a cross-sectional view taken along a horizontal plane extending through the head of a wearer showing the goggle of FIG. 1 worn with a helmet.

FIG. 7 is an isometric view showing the goggle of FIG. 1 worn with a helmet.

### DETAILED DESCRIPTION

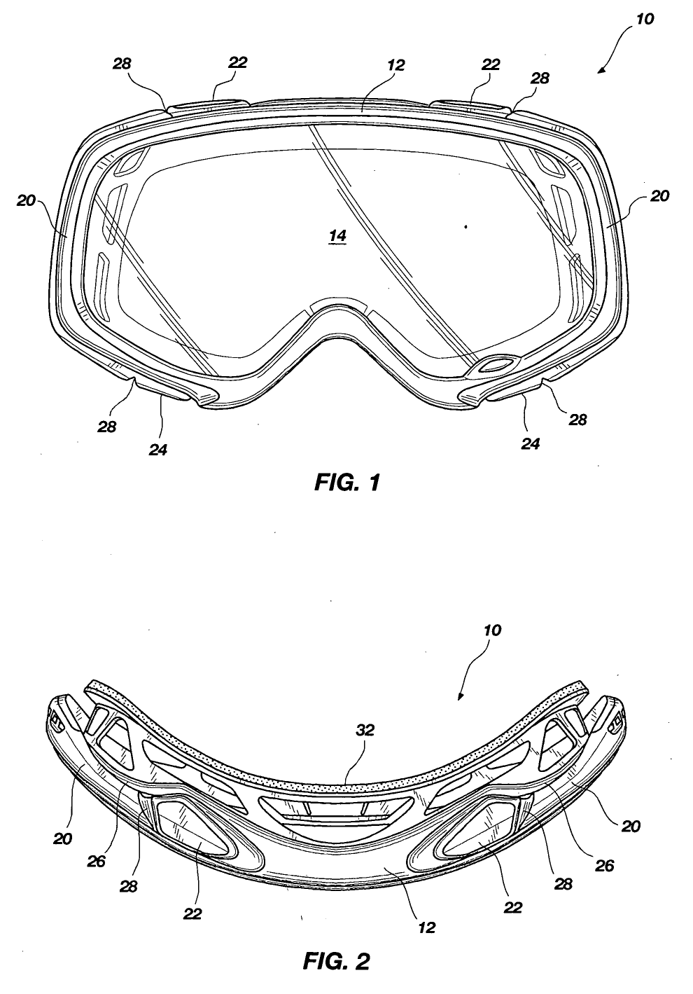

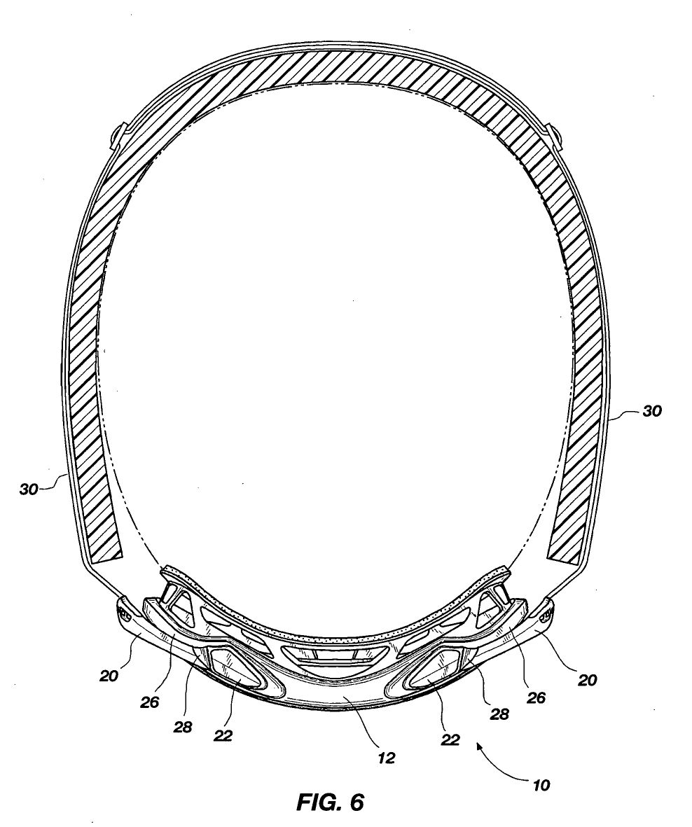

FIGS. 1-4 show one example of a goggle 10 in accordance with the invention. In this example, the goggle 10 includes a frame 12 having an opening for at least one lens 14 (FIGS. 1 and 4). The lens 14 may be a single layer, or it may be two or more lenses spaced apart from each other by a slight distance to reduce fogging. Also, goggles according to other examples of the invention may use two lenses, one of which is positioned in front of each eye as in typical spectacle-type goggles. Moreover, the frame 12 need not have the particular shape shown in FIGS. 1-4, but instead may have any suitable shape, such as that found in safety glasses, welding goggles or diving masks. The lens 14 is mounted in the frame 12 by having its edges received in a groove (not shown) extending around its inner periphery as is typically used for mounting such lenses. However, the lens of other goggles may be mounted to the frame by any suitable means.

The goggle 10 also includes headband attachment arms 20 having ends 22, 24 that are flexibly attached to upper and lower edges of the frame 12, respectively. The headband attachment arms 20 are fixedly attached to the frame 12 through flexures 28 formed by reduced thickness regions of the arms 20. However, since the arms may be made of a resilient material, such as the material used to form the frame 12, the flexures 28 are not required. Alternatively, the flexures 28 may be made of a material that is different from the material used to form the arms 20, or some other means of making a localized region more flexible may be used.

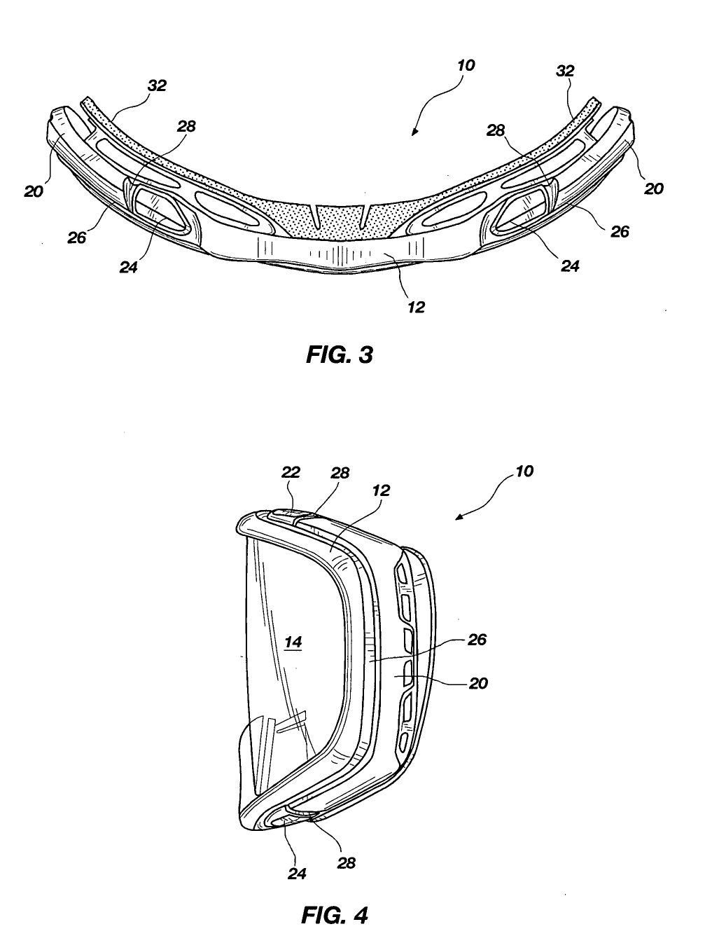

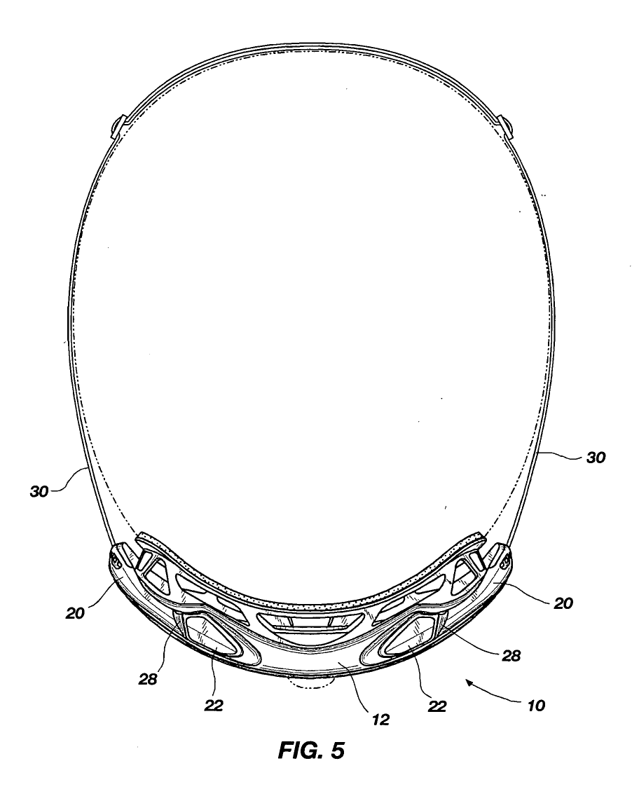

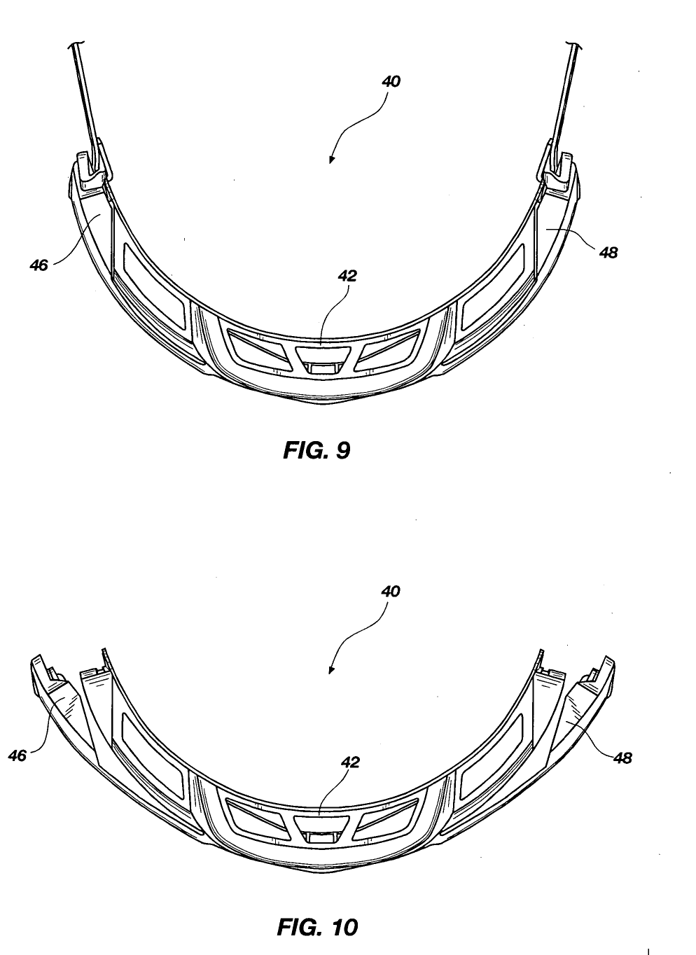

As best shown in FIG. 7, an elastic headband 30 may be secured to the arms 20 and used to hold the goggle 10 in place on a wearer's head. As shown in FIGS. 2 and 3, face gasket 32 may be provided to give a close fit of the goggle 10 to the wearer's face. Although the goggle 10 shown in FIGS. 1-4 uses the elastic headband 30 to hold the goggle 10 in place on the wearer's head, the goggle 10 may be held in place by other means. For example, a strap having tension adjustment buckles, earstems extending from the arms 20 to engage the sides and/or ears of a wearer, snaps, clips, hook-and-loop fasteners or other devices to secure the arms 20 to a helmet could be used. Also, although the elastic headband 30 is shown in FIGS. 1-4 as being fixedly secured to the center of the arms 20, in other embodiments the elastic headband 30 may be secured to the arms 20 at locations other than their centers.

The headband attachment arms 20 extend around the sides of the frame 12 from the top of the frame 12 to the bottom. Although not necessary, the headband attachment arms 20 may closely fit the contour of the outer periphery of the frame 12, and, as shown in FIGS. 2-4 and 6, may be received in recesses 26 formed in the frame 12 with shapes to match the shapes of the arms 20. As a result, the arms 20 and the frame 12 can form a smooth contour at the outer periphery around a side of the frame 12 so that the arms 20 are difficult to distinguish from the frame 12. By “smooth” in this context, it is meant that the frame 12 and arms 20 form a continuous shape without significant discontinuities, breaks or other irregularities in the shape, although the surfaces of the frame 12 and/or arms 20 may be rough or textured. Placing the arms 20 in the recesses 26 in this manner may also reduce the overall size of the goggle 10 and enhance the aerodynamic performance of the goggle 10.

One aspect of the goggle 10 illustrated in FIGS. 1-4 is that the attachment between the ends 22, 24 of the arms 20 and the frame 12 are located at a position spaced inwardly from the sides of the frame 12. As a result, and because of the rearwardly curved arcuate shape of the frame 12, the attachment of the arms 20 to the frame 12 is well forward of the sides of the frame 12 where the headband would normally be attached. The force of the headband 30 on the frame 12 therefore has a rearward component even if the headband 30 extends to the side and slightly to the rear, such as to locations directly across from the side edges of the frame 12. The frame 12 therefore remains in contact with the face of the wearer without any gap being created at the side edges of the goggle frame 12. In contrast, if the headband had extended to the sides of the frame 12 as is typical with conventional goggles, the frame 12 would be pulled from the face of the wearer when worn with a helmet, thereby creating a gap between the wearer's face and the edges of the frame.

In one example of the goggle 10, the arms 20 are formed separately from the frame 12, and they may be made from a material that is the same as or different from the material used to form the frame 12. The ends of the arms 20 are then attached to the frame 12 by suitable means, such as bonding. In another example of the goggle 10, the arms 20 and the frame 12 are integrally formed with each other as a unit. In either case, the frame 12 in the area where the arms 20 attach to the frame 12 may be relatively thin since there is no need for any portion of the arms 20 to project inwardly into the frame 12. Furthermore, there are very few constraints on the physical properties of the arms 12. For example, the arms 20 may be relatively soft and elastic like the material used to form the frame 12 even if the frame 12 and the arms 20 are not integrally formed with each other. There is no requirement that the arms 20 be sufficiently rigid to retain the shape of an attachment structure since the ends of the arms 20 need only be bonded to the outer surface of the frame. Of course, portions of the arms 20 may project into the frame 12 or vice-versa if desired, but it is not necessary to do so.

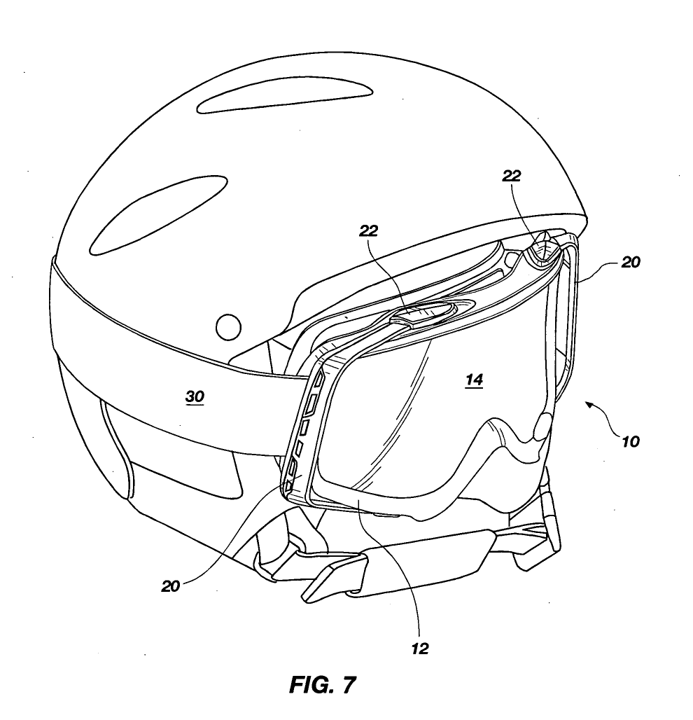

As shown in FIG. 5, when the goggle 10 is worn without a helmet, the arms 20 remain within the respective recesses 26 formed in the frame 12, and the headband 30 extends along the wearer's head. In this configuration, there would not be a problem holding the rear edge of the frame 12 against the face of the wearer.

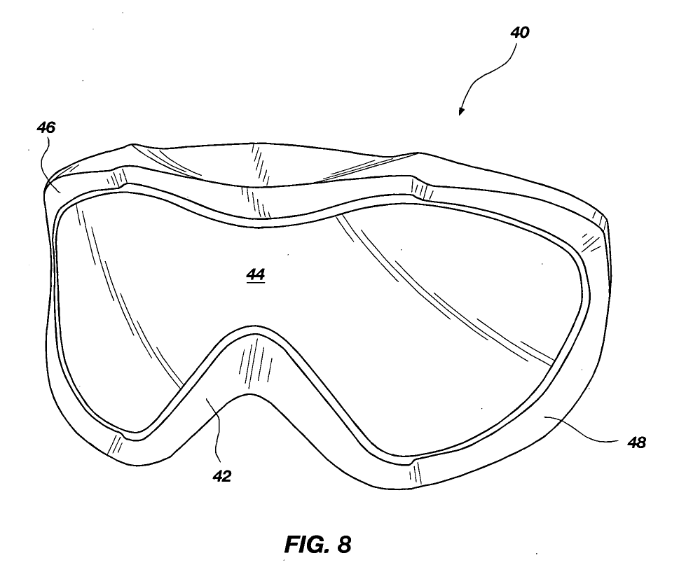

FIGS. 6 and 7 show the goggle 10 worn with a helmet H. If the arms 20 stayed in their position shown in FIG. 5, the elastic force of the headband 30 might easily pull the side edges of the frame 12 away from the face of the wearer. However, because the arms 20 are attached to the frame 12 inwardly and forwardly of the side edges of the frame 12, the headband 30 extends from the frame 12 to the side and in a slight rearward direction. As a result, the force exerted by the headband 30 exerts a rearward force on the frame 12 that holds the side edges of the frame 12 against the face of the wearer.

Although the present invention has been described with reference to the disclosed embodiments, persons skilled in the art will recognize that changes may be made in form and detail without departing from the spirit and scope of the invention. Such modifications are well within the skill of those ordinarily skilled in the art. Accordingly, the invention is not limited except as by the appended claims.

We claim:

1. A goggle comprising:
a frame having an a top and a bottom extending between opposite sides, and an outer periphery surrounding an opening, the frame having an arcuate, rearwardly curved configuration;
a lens mounted to the goggle frame;
a pair of generally U-shaped attachment arms extending along respective sides of the frame, each of the attachment arms having first and second ends fixedly attached to the top and bottom of the outer periphery of the frame at respective attachment locations that are spaced inwardly from the side of the frame, each of the arms having at least one resilient region structured to allow the arms to bend forwardly and rearwardly; and
a head support attached to the attachment arm.

2. The goggle of claim 1 wherein the outer periphery of the frame has a contour at the sides of the frame, and wherein the attachment arms together with the frame form a smooth contour at the outer periphery around the sides of the frame from the top of the frame to the bottom.

3. The goggle of claim 1 wherein the frame includes respective recesses in the outer periphery at a sides of the frame, each of the recesses receiving a respective one of the attachment arms.

4. The goggle of claim 1, further comprising a face gasket attached to the frame, the face gasket extending around an inner edge of the periphery of the frame.

5. The goggle of claim 4 wherein the attachment arms are fixedly attached to the top and bottom of the outer periphery of the frame at respective locations positioned forward of a rearward most portion of the face gasket.

6. The goggle of claim 4, further comprising a respective stop member formed at each side of the frame behind a respective one of the attachment arms, the stop members being structured to limit bending of the attachment arms rearwardly when the attachment arms abut the respective stop members.

7. The goggle of claim 6 wherein the attachments between the head support and respective attachment arms are forward of the face gasket when the arms are in their rearward-most positions with the attachment arms abutting the respective stop members.

8. The goggle of claim 1 wherein the attachment arms are fabricated of a resilient material so that the arms can bend in their entirety forwardly and rearwardly.

9. The goggle of claim 1 wherein the at least one resilient region comprises a reduced thickness region adjacent each end of the arms.

10. The goggle of claim 1 wherein the head support comprises a resilient headband having opposite ends attached to respective ones of the attachment arms.

11. The goggle of claim 1 wherein fastening pads are formed on each end of each of the attachment arms, and wherein the fastening pads are bonded to the top and bottom of the outer periphery of the frame.

12. The goggle of claim 1 wherein the attachment arms are integrally formed with the frame as a single unit.

13. A goggle and helmet assembly, comprising:
a helmet having a shell structured to partially surround the head of a wearer, the shell having a front cut-out defined by a pair of opposed side edges of the shell and an upper edge of the shell extending between the side edges of the shell;
a goggle, comprising:
an frame positioned within the front cut-out of the shell, the frame having an a top and a bottom extending between opposite sides, and an outer periphery surrounding an opening, the frame having an arcuate, rearwardly curved configuration;
a lens mounted to the goggle frame;
a pair of generally U-shaped attachment arms extending along respective sides of the frame, each of the attachment arms having first and second ends fixedly attached to the top and bottom of the outer periphery of the frame at respective sides of the frame at attachment locations that are spaced inwardly from the side of the frame, each of the arms having at least one resilient region structured to allow the arms to bend forwardly and rearwardly; and
an elastic headband having a pair of opposed ends attached to respective ones of the attachment arms, the headband extending around the rear of the shell from one side to the other, the elastic headband bending the arms forwardly in front of the frame to a position that causes the headband to extend outwardly and rearwardly from the attachment arms to the sides of the shell so that the headband exerts a rearward force on the frame.

14. The goggle and helmet assembly of claim 13 wherein the outer periphery of the frame has a contour at the sides of the frame, and wherein the attachment arms together with the frame form a smooth contour at the outer periphery around the sides of the frame from the top of the frame to the bottom.

15. The goggle and helmet assembly of claim 13 wherein the frame includes respective recesses in the outer periphery at a sides of the frame, each of the recesses receiving a respective one of the attachment arms.

16. The goggle and helmet assembly of claim 13, further comprising a face gasket attached to the frame, the face gasket extending around an inner edge of the periphery of the frame.

17. The goggle and helmet assembly of claim 16 wherein the attachment arms are fixedly attached to the top and bottom of the outer periphery of the frame at respective locations positioned forward of a rearward most portion of the face gasket.

18. The goggle and helmet assembly of claim 16, further comprising a respective stop member formed at each side of the frame behind a respective one of the attachment arms, the stop members being structured to limit bending of the attachment arms rearwardly when the attachment arms abut the respective stop members.

19. The goggle and helmet assembly of claim 18 wherein the attachments between the headband and respective attachment arms are forward of the face gasket when the arms are in their rearward-most positions with the attachment arms abutting the respective stop members.

20. The goggle and helmet assembly of claim 13 wherein the attachment arms are fabricated of a resilient material so that the arms can bend in their entirety forwardly and rearwardly.

21. The goggle and helmet assembly of claim 13 wherein the at least one resilient region comprises a reduced thickness region adjacent each end of the arms.

22. The goggle and helmet assembly of claim 13 wherein fastening pads are formed on each end of each of the attachment arms, and wherein the fastening pads are bonded to the top and bottom of the outer periphery of the frame.

23. The goggle and helmet assembly of claim 13 wherein the attachment arms are integrally formed with the frame as a single unit.

24. A method of wearing a goggle that with or without a helmet, the goggle including a frame having an a top and a bottom extending between opposite sides, and an outer periphery surrounding an opening in which a lens is mounted, the frame having an arcuate, rearwardly curved configuration, the goggle further including a pair of generally U-shaped resilient attachment arms extending along respective sides of the frame with their ends fixedly attached to the top and bottom of the outer periphery of the frame at respective sides of the frame at attachment locations that are spaced inwardly from the side of the frame, and an elastic headband connected between the attachment arms, the method comprising:
when the goggle is worn with a helmet, wearing the goggle so that the headband extends from the attachment arms substantially laterally and rearwardly at a second angle that is substantially forward of the first angle, thereby bending the attachment arm rearwardly to a second position that is forward of the first position.

25. The method of claim 24 wherein the frame includes respective recesses in the outer periphery at a sides of the frame, and wherein the attachment arms are received within the respective recesses when the goggle is worn without a helmet so that the attachment arms are in the first position.

26. The method of claim 25 wherein the frame includes respective recesses in the outer periphery at a sides of the frame, and wherein the attachment arms are removed from the respective recesses when the goggle is worn with a helmet so that the attachment arms are in the second position.

27. The method of claim 24, further comprising a face gasket extending around an inner edge of the periphery of the frame, and wherein the attachment arms are fixedly attached to the top and bottom of the outer periphery of the frame at respective locations positioned forward of a rearward most portion of the face gasket.

28. The method of claim 24, further comprising a respective stop member formed at each side of the frame behind a respective one of the attachment arms to limit bending of the attachment arms rearwardly when the attachment arms abut the respective stop members, and wherein the stop members abut the respective stop members when the goggle is worn without a helmet so that the attachment arms are in the first position.

29. The method of claim 28 wherein the attachments between the head support and respective attachment arms are forward of the face gasket when the goggle is worn without a helmet so that the attachment arms are in the first position.

30. The method of claim 24 wherein the attachment arms are fabricated entirely of a resilient material so that the arms can bend in their entirety forwardly and rearwardly.

31. The method of claim 24 wherein the attachment arms each include a pair of resilient flexures located adjacent opposite ends of the attachment arms.

32. The method of claim 24 wherein fastening pads are formed on each end of each of the attachment arms, and wherein the fastening pads are bonded to the top and bottom of the outer periphery of the frame.

33. The goggle of claim 24 wherein the attachment arms are integrally formed with the frame as a single unit.
